(19) United States
(12) Patent Application Publication
(10) Pub. No.: US 2002/0148469 A1
(43) Pub. Date: Oct. 17, 2002

(54) Inhaler

(76) Inventors: David O'Leary, Grays (GB); Brian Barney, Hatfield Broad Oak (GB); Andrew Dickson, Harlow (GB)

Correspondence Address: BEYER WEAVER & THOMAS LLP, P.O. BOX 778, BERKELEY, CA, 94704-0778, US

(21) Appl. No.: 09/834,409

(22) Filed: Apr. 12, 2001

(51) Int. Cl. B05D 7/14; A61M 15/00; A61M 16/00; B65D 83/06

(52) U.S. Cl. 128/203.15

(57) ABSTRACT

A dispensing system includes a vessel having an exit port in a first end wall and an open second end, a dispensing member capable of adopting a receiving position at which the exit port is open and discharge position at which the exit port is closed, the dispensing member having a substantially flat upper surface at least a part of which is in continuous sealing contact with the first end wall of the vessel, and an actuator for driving a proportion of a volume of gas through a powdered formulation to the exit port and into the metering chamber of the dispensing member in the receiving position.

The present invention relates to an improved metered dose inhaler for administering a discrete amount of a formulation (eg a powdered formulation) to a subject, in particular to improvements in the dispensing system of a metered dose powder inhaler.

Portable aids are widely available to a subject wishing to self-administer therapeutic and preventative formulations to combat the symptoms of a respiratory disorder such as asthma. Such aids are generally arranged to dispense a discrete amount of the formulation (usually in the form of a fluid or particulate medicament entrained in a stream of gas or vapour) into the respiratory passages and are widely referred to as metered dose inhalers.

Typically, a metered dose inhaler comprises two main parts, namely a dispensing system for dispensing the discrete amount (ie the metered dose) of formulation and a delivery system for delivering the metered dose of formulation to the subject. The present invention is concerned primarily with improvements in the dispensing system and is not intended to be limited to any particular delivery system.

The dispensing systems of many conventional metered dose powder inhalers have various features in common. Powdered formulation and an amount of air is housed in a hopper. By applying a compressive force to an actuator at an end of the hopper, air is driven through the powdered formulation and an air/powder mixture passes through an exit port in the basal wall of the hopper. The air/powder mixture passes into a metering chamber, the volume of which defines the size of the metered dose. As the air pressure in the dispensing system increases during the dispensing operation, a passage is opened to vent air from the metering chamber into the atmosphere. Following dispensing of the air/powder mixture, the metered dose is introduced into a delivery system from which it is delivered to the subject.

A specific example of a metered dose powder inhaler with a dispensing system of this type is illustrated in U.S. Pat. No. 5,503,144 (Norton Healthcare Limited). The dispensing system of the metered dose powder inhaler described in U.S. Pat. No. 5,503,144 has a metering chamber incorporated in a plate which is positioned remote from the basal wall of the hopper. During dispensing of the powder/air mixture into the metering chamber, the gap between the basal wall of the hopper and the plate is sufficient to withstand powder escape whilst providing a vent to atmosphere.

When a metered dose powder inhaler is operated in this manner, the air/powder mixture undergoes rapid initial expansion at atmospheric pressure into the relatively large volume of the metering chamber. Characteristics of the resulting airflow in the metering chamber (eg the velocity, direction and turbulence) are thought to determine the extent to which the powdered formulation deagglomerates. At least a degree of deagglomeration is desirable to produce fine particles of powdered formulation which are capable of entering into the lung where they can have their desired effect. Insufficient deagglomeration leaves bulky particles which tend to accumulate in the throat and upper part of the lungs where they are unable to have their desired effect and may have undesirable, localised side effects.

The present invention is based on the recognition that the quality of a metered dose of powdered formulation dispensed by a metered dose powder inhaler may be improved by providing an otherwise sealed metering chamber with an isolated volume adapted to bleed the metering chamber.

Thus viewed from one aspect the present invention provides a metered dose powder inhaler for administering a metered dose of a powdered formulation to a subject, said metered dose powder inhaler having a dispensing system and a delivery system, wherein:

(1) the dispensing system comprises:

a vessel having an exit port in a first end wall and an open second end, said vessel containing the powdered formulation and a volume of gas (eg air) between the second end and the powdered formulation;

a dispensing member capable of adopting a receiving position at which the exit port is open and a discharge position at which the exit port is closed, said dispensing member having a substantially flat upper surface at least a part of which is in continuous sealing contact with the first end wall of the vessel, wherein the substantially flat upper surface incorporates (1) a metering chamber of a size sufficient to receive a metered dose of powdered formulation in the receiving position and (2) a discontinuity which in the receiving position defines an isolated volume adapted to bleed the metering chamber; and

an actuator for driving a proportion of the volume of gas through the powdered formulation to the exit port and into the metering chamber of the dispensing member in the receiving position, and

(2) the delivery system is in fluid communication with the dispensing member in the discharge position so as to deliver the metered dose to the subject.

Although the incorporation of a discontinuity in the substantially flat upper surface of the dispensing member in sealing contact with the first end wall of the vessel might have been expected to lead to certain disadvantages (such as powder escape and clogging), it has been surprisingly found that by defining a volume in fluid communication solely with the metering chamber, the discontinuity in the substantially flat upper surface actually improves the quality of the metered dose of formulation dispensed to the subject. Without wishing to be bound by any theoretical considerations, it is thought that by regulating the rate at which gas escapes from the metering chamber, the discontinuity controls the deagglomeration of the powder/gas mixture. Typically, the fine particle dose is improved by an amount in the range 30 to 34 wt %.

In a preferred embodiment, the dispensing member is rotationally or (preferably) linearly moveable between the receiving position and the discharge position. Preferably the dispensing member is slidably moveable between the receiving position and the discharge position (so as to maintain at least a part of the substantially flat upper surface in continuous sealing contact with the first end wall of the vessel).

Although the gas in the vessel may be any inert gas (such as air or nitrogen), air is preferred.

Preferably the dispensing member is a dispensing plate (eg a rectangular dispensing plate).

In a preferred embodiment, the substantially flat upper surface of the dispensing member incorporates a discontinuity being an indentation or depression in fluid communication solely with the metering chamber.

The precise shape of the discontinuity (eg indentation or depression) manufactured in the substantially flat upper surface is not thought to have a bearing on the whether or not the desired effect is achieved. Nevertheless it is preferred that the discontinuity is smooth (eg without internal ledges or corners)

In a preferred embodiment, the discontinuity defines a substantially regular, isolated volume. For example, the discontinuity may define a substantially annular, isolated volume.

Preferably, the discontinuity is at or near to the lip of (and in fluid communication solely with) the metering chamber. Particularly preferably, the discontinuity is in continuous circumferential fluid communication with the metering chamber. For example, the discontinuity may surround the lip of the metering chamber.

In a preferred embodiment, the discontinuity is a shallow discontinuity (eg a shallow indentation or depression). Typically, the depth of the discontinuity is sufficiently low to minimise powder escape from the metering chamber but sufficiently great to exert the desired effect. For example, the depth of the shallow discontinuity may be in the range 20 to 40 microns.

In a preferred embodiment, the metering chamber is partially spherical (eg hemispherical).

In a preferred embodiment, the internal walls of the vessel are shaped to assist flow of powdered formulation. Particularly preferably, the internal walls of the vessel are non-uniformly tapered inwardly to the exit port. For example, the internal walls of the vessel may taper inwardly to a narrow bore connected to the exit port.

In an embodiment of the invention, the actuator is operable by a compressive force applied to the air at or near to the open second end of the vessel. For example, an axial compressive force may be applied to the air at or near to the open second end. Typically a low compressive force is required.

In a preferred embodiment, the actuator comprises: flexible bellows fixed at or near to the open second end of the vessel and compressing means for compressing the bellows. The flexible bellows may be fixed at or near to the open second end of the vessel in any convenient manner. For example, a first end of the flexible bellows may be retained on a retaining means (such as an annular retaining shoulder) on the vessel. Preferably the compressing means is a spring secured to the second end of the flexible bellows. A filter at or near to the open second end of the vessel may be used to ensure that air expelled from the bellows is clean and that powdered formulation does not enter the bellows (where it might interfere with correct functioning).

In a preferred embodiment, the dispensing system further comprises urging means for maintaining at least a part of the substantially flat upper surface in continuous sealing contact with the first end wall of the vessel. This advantageously ensures that at all positions other than the receiving position the exit port is sealingly closed.

In a preferred embodiment, the metered dose powder inhaler of the invention is operable substantially vertically (preferably with the open second end of the vessel uppermost).

The delivery system may take any convenient structure. The metered dose may be released from the metering chamber in the discharge position by air flow and/or mechanically. The air flow may be instigated by the inspiration of the subject. The delivery system comprises a flow path between the metering chamber in the discharge position and the subject (eg a flow path defined by one or more inhaling chambers or passageways). Typically the delivery system terminates in a delivery outlet suitable for the desired mode of administration.

The metered dose powder inhaler of the invention may be of the manually operable or breath actuated type. It is thought that pulmonary inhalation would be the primary application of the invention either nasally or (preferably) orally and the delivery outlet of the delivery system may be adapted accordingly.

The present invention is suitable for administration of a powdered active ingredient (eg a powdered therapeutic or preventative medicament) or an active ingredient mixed in a formulation with a suitable carrier such as lactose.

Viewed from a further aspect the present invention provides a dispensing member as hereinbefore defined.

The present invention will now be described in a non-limitative sense with reference to the accompanying figures in which:

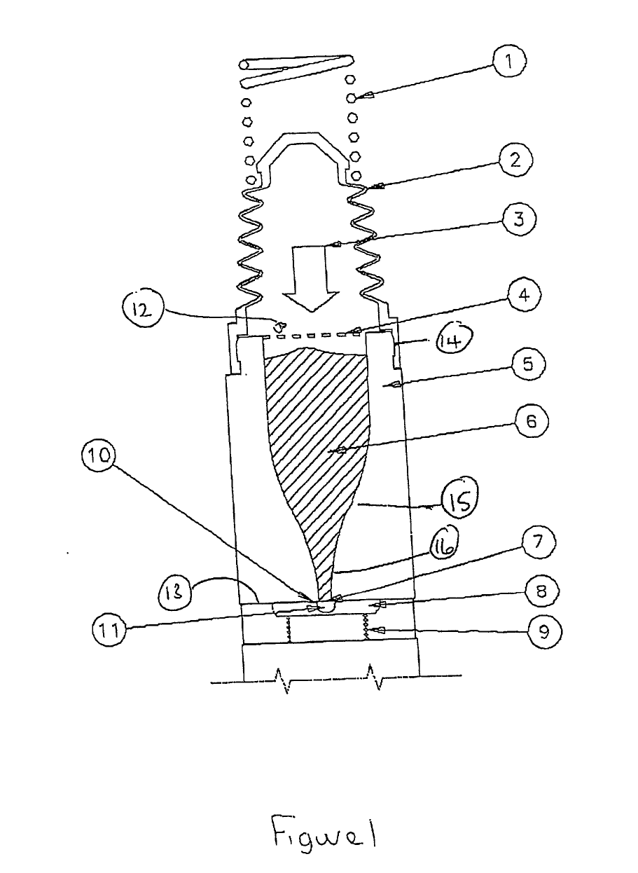

FIG. 1 illustrates schematically part of an embodiment of the metered dose powder inhaler of the invention in cross-section; and

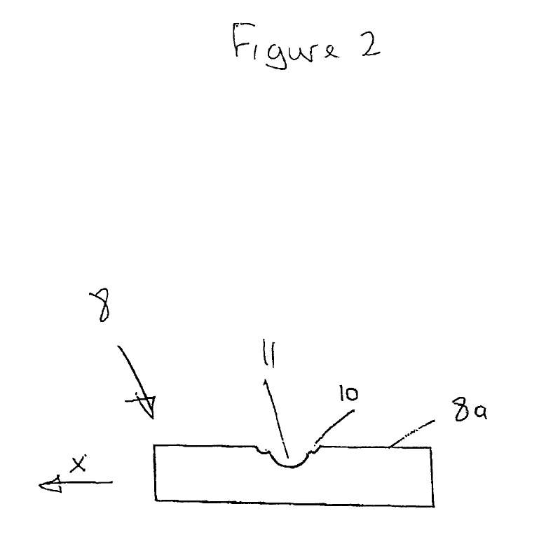

FIG. 2 illustrates an exploded cross-sectional view of the dispensing plate of an embodiment of the metered dose powder inhaler of the invention.

In FIG. 1, there is illustrated a partial, cross-sectional view of an embodiment of the metered dose powder inhaler of the invention. A hopper 5 comprises a first end wall 13 in which there is an exit port 7 and an open second end 12 and houses a bed of finely powdered formulation 6 beneath an amount of air. The internal walls 15 taper inwardly and non-uniformly to a narrow bore 16 connected to the exit port 7.

At the open second end 12 of the hopper 5 is disposed an actuator. The actuator comprises flexible bellows 2 retained coaxially on an annular retaining shoulder 14 of hopper 5. At the opposite end, the bellows 2 are connected to a main spring 1. The main spring 1 may be lightly compressed to axially compress the flexible bellows 2 in order to drive a proportion of air in direction 3 towards the open second end 12 of the hopper 5. Across the open second end 12 is provided an air filter 4. The internal walls 15 and narrow bore 16 of the hopper 5 are adapted to permit powdered formulation to flow evenly towards the exit port 7. The proportion of air is driven through the bed of powdered formulation 6 to release a blend of air/powdered formulation through exit port 7.

A slide plate 8 is illustrated in the receiving position in FIG. 1 and is illustrated alone in exploded view in FIG. 2. The upper surface 8a of the slide plate 8 is substantially flat and in sealing contact with the first end wall 13 of the hopper 5. Sealing contact is maintained by virtue of sealing spring 9 which urges the upper surface 8a of slide plate 8 against the first end wall 13 of the hopper 5. Incorporated in the upper surface 8a is a metering chamber 11 of substantially hemispherical volume and an annular, shallow depression 10 surrounding the lip of and in fluid communication solely with the metering chamber 11. In the receiving position, the blend of air/powdered formulation is released through exit port 7 into the metering chamber 11 of the slide plate 8 which lies directly beneath the exit port 7. The metering chamber 11 of the slide plate 8 has a volume which defines the dose of formulation to be delivered to the subject. The isolated, shallow depression 10 serves to vent the air from the metering chamber 11 and improve the quality of the metered dose of formulation.

From the receiving position shown in FIG. 1, the slide plate 8 is capable of slidably moving (in direction X) to a discharge position so as to maintain at least a part of the substantially flat upper surface 8a of the slide plate 8 in continuous sealing contact with the first end wall 13 of the hopper 5. In the discharge position, the metered dose of formulation is discharged into a delivery system for inhalation and the exit port 7 is closed The movement of the slide plate from the receiving position to the discharge position and the subsequent action of the delivery system is described elsewhere (for example in EP-A-0561838). Typically the movement of the slide plate is controlled by a spring.

1. A metered dose powder inhaler having a dispensing system and a delivery system, the delivery system comprising:
a vessel having an exit port in a first end wall and open second end, said vessel containing a powdered formulation and a volume of gas between the second end and the powdered formulation;
a dispensing member capable of adopting a receiving position at which the exit port is open and a discharge position at which the exit port is closed, said dispensing member having a substantially flat upper surface at least a part of which is in continuous sealing contact with the first end wall of the vessel, wherein the substantially flat upper surface incorporates (a) a metering chamber of a size sufficient to receive a metered dose of the powdered formulation in the receiving position and (b) a discontinuity which in the receiving position defines an isolated volume adapted to bleed the metering chamber; and
an actuator for driving a proportion of the volume of gas through the powdered formulation to the exit port and into the metering chamber of the dispensing member in the receiving position.

2. The inhaler of claim 1, wherein the delivery system is in fluid communication with the dispensing member in the discharge position so as to deliver the metered dose to the subject.

3. A dispensing system for dispensing a discrete amount of a formulation to a delivery system of an inhaler, the dispensing system comprising:
a hopper for storing said formulation;
a metering chamber fluidly coupled to said hopper for receiving a formulation-air mixture; and
an isolated volume adapted to bleed the metering chamber.
